(19) United States
(12) Patent Application Publication
(10) Pub. No.: US 2003/0056384 A1
(43) Pub. Date: Mar. 27, 2003

(54) Glove sizing system

(76) Inventor: Francis J. Masley, Wilmington, DE (US)

Correspondence Address: FRANCIS J. MASLEY, 222 WAVERLY ROAD, WILMINGTON, DE, 19803, US

(21) Appl. No.: 09/927,232

(22) Filed: Sep. 25, 2001

(51) Int. Cl. A61B 5/107

(52) U.S. Cl. 33/512

(57) ABSTRACT

An improved glove sizing system is provided which is defined by a utilization of a two-dimensional dimension of a human hand in a glove size designation (64). The said glove size designation applies to a standard glove size and a plurality of the said standard glove sizes represent a glove system (66) suitable for large-scale production. The said glove size designation (64) facilitates a quantified and logical process for a user to select a proper glove size for best-fit and tactility. Additionally, the glove sizing system correlates to dimensions measured easily with a common ruler or scale.

### BACKGROUND—FIELD OF INVENTION

This invention relates to gloves, specifically an improved glove sizing system that facilitates a quantified process for a user to select a proper glove size for best fit and tactility.

### BACKGROUND—DESCRIPTION OF PRIOR ART

There has been a long-felt but unsolved need for better fitting and more tactile gloves in the end use areas of firefighting and military. In these end use applications the user often requires protection for their hands. Such protection typically necessitates a relatively thick and non-stretchable glove. At the same time the user has a need to perform tasks that require manipulation of relatively small objects such as switches and buttons such as those found on radios, phones and computers. The act of manipulating small objects with one's hands and the sense of touch are often referred to as tactility. It is known that there is an inverse relationship between tactility and the thickness of the glove or hand covering at the fingertips. Tactility is diminished as the distance increases between the skin surface of the fingertip and the outer surface of the hand covering.

Therefore it is advantageous to the user to select and wear hand coverings and/or gloves that minimize the amount of excess material at the fingertips. This is especially the case with the index finger or digit #2 because this finger is most often used in the manipulation of small objects. While the user has recognized the need for a close fit at the fingertip, there has not been a glove sizing system developed that provides a quantitative solution for a user to select gloves with the proper fingertip lengths.

Currently used and previously available glove sizing systems have utilized standard sizing designations. The deficiencies of these designations are that they relate either to hand dimensions obtained from specialized hand-measuring devices or they relate only in an abstract and general way to actual hand dimensions. When an abstract size designator is utilized, there is no quantified correlation between the size designator and the actual dimensions of a human hand for which the glove is intended to fit.

Previously published patents and current in use glove sizing systems have deficiencies associated with specialized hand measuring devices and abstract size descriptors.

In U.S. Pat. No. 2,146,799 to Davis (1939) and U.S. Pat. No. 2,176,288 to Baird et al (1939), a standard size designation is based on a hand circumference measurement and the measurement is dependent on one using a special hand-measuring device. The dependency on this measuring device is a limiting feature to the person who does not have the device readily available.

U.S. Pat. No. 1, 997,920 to Bliss (1935), U.S. Pat. No. 2,605,548 to Clarke (1952), and U.S. Pat. No. 4,173,074 to Newman et al (1979) illustrate special measuring devices that determine an arbitrary or abstract size designation. The abstract size designations are only of value to people who have access to the special hand-measuring devices illustrated.

U.S. Pat. No. 4,360,972 to Montgomery (1982) teaches a method for measuring the hand on a special chart and applying an abstract descriptor to the standard sizes as follows; LL, LR, ML, MR, SL, SR for Large Long, Large Regular, Medium Long, Medium Regular, Small Long, and Small Regular respectively. Again, the abstract size designations are only of value to a person who has access to the special hand-measuring chart illustrated.

U.S. Pat. No. 4,897,924 to Tepley (1990) illustrates and describes a special apparatus for measuring dimensions of a human hand. These dimensions are then used for the manufacturing of custom sized gloves. However, no attempt is made to develop standard glove sizes for large-scale production.

Regarding sizing systems currently in use, a standard size is defined by an arbitrary descriptor or an abstract size designation such as a word, alpha or numeric designation. Examples of said designations include the following: small, medium, large, x-large, etc., or A, B, C, etc., or 1, 2, 3, etc. These abstract designations provide only a qualitative size comparison to a user that is trying to select appropriately sized gloves for their hands.

Prior Art—FIG. 1

FIG. 1 is a table listing examples of current in use glove sizing systems with their standard abstract sizing designations 10. The abstract sizing designations are listed in rank order to provide a rough correlation amongst the different sizing systems. The largest size is listed at the top of the table and the smallest size at the bottom. Within a specific sizing system, it can be observed that there is no quantitative correlation between the abstract sizing designations and dimensions of a human hand. Therefore, a qualitative process and/or a special hand-measuring device must be utilized for a person or user selecting an appropriate glove size for their hand.

Additionally, there are times when a person or a group of people are required to procure and use several different styles of gloves made by different manufacturers. It is possible that the user would be presented with several of the glove sizing systems listed in FIG. 1. Unfortunately, in addition to the abstract sizing designations having no quantified correlation with human hand dimensions, they also have no quantified correlation with each other. This makes the task of selecting an appropriate glove size for each of the different styles of gloves a qualitative process. This qualitative selection process becomes a trial and error process. A user must go through the process of trying on and taking off gloves, also referred to as donning and doffing gloves, till the appropriate glove size is found. This process works best if new samples of all the glove sizes are provided to the user, however, this is not always practical.

Prior Art—NFPA Standard 1971

NFPA Standard 1971, incorporated by reference, is administered by the National Fire Protection Association, Inc. Special glove sizing requirements are included in the NFPA 1971 Standard on Protective Ensemble for Structural Fire Fighting 2000 Edition. These special sizing requirements provide insight into the complexity surrounding the task of designing gloves for proper fit and tactility. Additionally the task of labeling a designed glove size with an appropriate glove size designation can also be ambiguous and uncertain. This is due to the multiple abstract size designations that are possible due to the overlapping ranges for hand dimensions.

In the NFPA Standard 1971, a table listing specific hand dimensions is provided for each required glove size. An understanding of how glove size designations are correlated to actual human hand dimensions can best be made by referencing the tables provided in Section 4-3.5.3 (a) through (e) of the NFPA Standard 1971. These tables are incorporated by reference below.

TABLE 4-3.5.3(a)
Sizing for Extra-Small (XS) Glove

| | Cm | In. |
|---|---|---|
| Range for hand length: | 16.25-17.25 | 6.40-6.79 |
| Range for hand circumference: | 16.25-20.25 | 6.40-7.97 |

| | | | Range to Be | |
|---|---|---|---|---|
| | Mid-Size Value | | Accommodated | |
| | Cm | In. | Cm | In. |
| Digit 1 circumference | 6.17 | 2.43 | 5.60-6.74 | 2.20-2.65 |
| Digit 2 circumference | 6.06 | 2.39 | 5.50-6.63 | 2.17-2.61 |
| Digit 3 circumference | 6.08 | 2.39 | 5.53-6.63 | 2.18-2.61 |
| Digit 4 circumference | 5.69 | 2.24 | 5.12-6.26 | 2.02-2.46 |
| Digit 5 circumference | 5.00 | 1.97 | 4.48-5.52 | 1.76-2.17 |
| Digit 1 Length | 4.94 | 1.94 | 4.36-5.52 | 1.72-2.17 |
| Digit 2 Length | 6.44 | 2.54 | 5.75-7.12 | 2.26-2.80 |
| Digit 3 Length | 7.29 | 2.87 | 6.71-7.87 | 2.64-3.10 |
| Digit 4 Length | 6.78 | 2.67 | 6.13-7.42 | 2.41-2.92 |
| Digit 5 Length | 5.09 | 2.00 | 4.52-5.66 | 1.78-2.23 |
| Hand Circumference | 18.25 | 17.19 | 16.34-20.16 | 6.43-7.94 |
| Hand Length | 16.75 | 6.59 | 16.27-17.23 | 6.41-6.78 |

TABLE 4-3.5.3(b)
Sizing for Small (S) Glove

| | Cm | In. |
|---|---|---|
| Range for hand length: | 17.25-18.25 | 6.79-7.19 |
| Range for hand circumference: | 17.25-21.25 | 6.79-8.37 |

| | | | Range to Be | |
|---|---|---|---|---|
| | Mid-Size Value | | Accommodated | |
| | Cm | In. | Cm | In. |
| Digit 1 circumference | 6.40 | 2.52 | 5.82-6.97 | 2.29-2.74 |
| Digit 2 circumference | 6.29 | 2.48 | 5.73-6.85 | 2.26-2.70 |
| Digit 3 circumference | 6.31 | 2.48 | 5.76-6.87 | 2.27-2.70 |
| Digit 4 circumference | 5.92 | 2.33 | 5.35-6.49 | 2.11-2.56 |
| Digit 5 circumference | 5.22 | 2.06 | 4.70-5.74 | 1.85-2.26 |
| Digit 1 Length | 5.31 | 2.09 | 4.74-5.89 | 1.87-2.32 |
| Digit 2 Length | 6.89 | 2.71 | 6.21-7.57 | 2.44-2.98 |
| Digit 3 Length | 7.71 | 3.04 | 7.13-8.30 | 2.81-3.27 |
| Digit 4 Length | 7.19 | 2.83 | 6.55-7.03 | 2.58-3.08 |
| Digit 5 Length | 5.44 | 2.14 | 4.87-6.01 | 1.92-2.37 |
| Hand Circumference | 19.25 | 7.58 | 17.34-21.16 | 6.83-8.33 |
| Hand Length | 17.75 | 6.99 | 17.27-18.23 | 6.80-7.18 |

TABLE 4-3.5.3(c)
Sizing for Medium (M) Glove

| | Cm | In. |
|---|---|---|
| Range for hand length: | 18.25-19.25 | 7.19-7.58 |
| Range for hand circumference: | 18.25-22.25 | 7.19-8.76 |

| | | | Range to Be | |
|---|---|---|---|---|
| | Mid-Size Value | | Accommodated | |
| | Cm | In. | Cm | In. |
| Digit 1 circumference | 7.01 | 2.76 | 6.36-7.65 | 2.50-3.01 |
| Digit 2 circumference | 6.82 | 2.69 | 6.31-7.32 | 2.48-2.88 |
| Digit 3 circumference | 6.83 | 2.69 | 6.26-7.40 | 2.46-2.91 |
| Digit 4 circumference | 6.34 | 2.50 | 5.78-6.90 | 2.28-2.72 |
| Digit 5 circumference | 5.63 | 2.22 | 5.09-6.17 | 2.00-2.43 |
| Digit 1 Length | 5.63 | 2.22 | 5.00-6.26 | 1.97-2.46 |
| Digit 2 Length | 7.11 | 2.80 | 6.50-7.72 | 2.56-3.04 |
| Digit 3 Length | 8.07 | 3.18 | 7.55-8.58 | 2.97-3.38 |
| Digit 4 Length | 7.61 | 3.00 | 7.14-8.08 | 2.81-3.18 |
| Digit 5 Length | 5.78 | 2.28 | 5.16-6.41 | 2.03-2.52 |
| Hand Circumference | 20.25 | 7.97 | 18.34-22.16 | 7.22-8.72 |
| Hand Length | 18.75 | 7.38 | 18.27-19.23 | 7.19-7.57 |

TABLE 4-3.5.3(d)
Sizing for Large (L) Glove

| | Cm | In. |
|---|---|---|
| Range for hand length: | 19.25-20.25 | 7.58-7.97 |
| Range for hand circumference: | 19.25-23.25 | 7.58-9.15 |

| | | | Range to Be | |
|---|---|---|---|---|
| | Mid-Size Value | | Accommodated | |
| | Cm | In. | Cm | In. |
| Digit 1 circumference | 7.26 | 2.86 | 6.62-7.91 | 2.61-3.11 |
| Digit 2 circumference | 7.03 | 2.77 | 6.53-7.54 | 2.57-2.97 |
| Digit 3 circumference | 7.10 | 2.80 | 6.53-7.66 | 2.57-3.02 |
| Digit 4 circumference | 6.60 | 2.60 | 6.04-7.16 | 2.38-2.82 |
| Digit 5 circumference | 5.85 | 2.30 | 5.31-6.39 | 2.09-2.52 |
| Digit 1 Length | 5.87 | 2.31 | 5.24-6.50 | 2.06-2.56 |
| Digit 2 Length | 7.49 | 2.95 | 6.88-8.10 | 2.72-3.19 |
| Digit 3 Length | 8.54 | 3.36 | 8.03-9.06 | 3.16-3.57 |
| Digit 4 Length | 8.03 | 3.16 | 7.56-8.50 | 2.98-3.35 |
| Digit 5 Length | 6.13 | 2.41 | 5.51-6.75 | 2.17-2.66 |
| Hand Circumference | 21.25 | 8.37 | 19.34-23.16 | 7.61-9.12 |
| Hand Length | 19.75 | 7.78 | 19.27-20.23 | 7.59-7.96 |

TABLE 4-3.5.3(e)
Sizing for Extra-Large (XL) Glove

| | Cm | In. |
|---|---|---|
| Range for hand length: | 20.25-21.25 | 7.97-8.37 |
| Range for hand circumference: | 20.25-24.25 | 7.97-9.55 |

| | | | Range to Be | |
|---|---|---|---|---|
| | Mid-Size Value | | Accommodated | |
| | Cm | In. | Cm | In. |
| Digit 1 circumference | 7.52 | 2.96 | 6.87-8.16 | 2.70-3.21 |
| Digit 2 circumference | 7.25 | 2.85 | 6.74-7.76 | 2.65-3.06 |
| Digit 3 circumference | 7.36 | 2.90 | 6.79-7.93 | 2.67-3.12 |
| Digit 4 circumference | 6.86 | 2.70 | 6.30-7.42 | 2.48-2.92 |
| Digit 5 circumference | 6.06 | 2.39 | 5.25-6.60 | 2.17-2.60 |
| Digit 1 Length | 6.11 | 2.14 | 5.48-6.75 | 2.16-2.66 |
| Digit 2 Length | 7.86 | 3.09 | 7.26-8.47 | 2.86-3.33 |
| Digit 3 Length | 9.02 | 3.55 | 8.51-9.54 | 3.35-3.76 |
| Digit 4 Length | 8.44 | 3.32 | 9.97-8.91 | 3.14-3.51 |
| Digit 5 Length | 6.48 | 2.55 | 5.85-7.10 | 2.30-2.80 |
| Hand Circumference | 22.25 | 8.76 | 20.34-24.16 | 8.01-9.51 |
| Hand Length | 20.75 | 8.17 | 20.27-21.23 | 7.98-8.36 |

In the tables, ranges for hand dimensions are given. These ranges are used for determining an appropriate glove size designation to be indicated on a label that is attached to the glove. Five size ranges are given and labeled as XS, S, M, L, and XL for Extra-Small, Small, Medium, Large, and Extra-Large respectively. It is the choice of the manufacturer to provide as many sizes as they desire provided that a minimum of five glove sizes is offered. The standard specifies that the glove sizes offered should be labeled with one of the designations noted in the tables above and that the glove shall fit the hand dimension ranges specified.

From a broad perspective, it appears that the two overall hand dimension ranges for hand length and hand circumference provide a quantified basis for relating a designated glove size to one's hand dimensions. However, upon closer inspection of the hand dimension ranges, it is evident that the user will not have a quantified basis for selecting the appropriate glove size for their specific finger length dimensions. This is especially true in regards to a user selecting the optimal glove size for performance requiring tactility.

Upon close inspection of the NPFA hand dimension ranges it is noted that there is considerable overlap in the individual finger length dimensions. Specifically there is considerable overlap in the index finger length dimension referred to as digit 2. This considerable overlap in the index finger length dimension creates great ambiguity when the glove manufacturer develops their patterns for a specific glove size designation. Surprisingly, it is possible as shown in the following example, that a glove labeled with a size designation “S” for Small could have the same pattern dimension for the index finger length as a glove labeled with a size designation “XL” for Extra-Large.

For example, the dimension range for the digit 2 finger length or index finger length for size S or Small is 6.21 cm to 7.57 cm while the dimension range for the digit 2 finger length for size XL or Extra-Large is 7.26 cm to 8.47 cm. Therefore it is possible for two gloves, one designated as “S” and one designated as “XL” to have the same pattern dimension of 7.5 cm for the digit 2 finger length.

Prior Art—FIG. 2

Shown in FIG. 2 is a chart of the US Military Unisex Dress Glove Size Designations vs. Pattern Dimensions. These dimensions have been measured from military specification drawings provided for gloves made according to military specification A-A-50386. In FIG. 2 it can be observed that abstract sizing designations of 1, 1C, 2, 2C, 3, 3C, 4, 4C, 5, 5C, 6, 6C, 7, 7C, 8, 9, and 10 are utilized to denote a glove size. A “C” is used to correspond to a “cadet” finger length.

A total of 17 size designations and the corresponding pattern dimensions are graphed. The relative similarities and differences in the pattern dimensions are shown graphically to illustrate the likely confusion confronting a person who is trying to select an optimally sized glove for their hand. Typically the user would only have knowledge of the size designations available and a bin or container of different size gloves when beginning their search for a standard glove size that has an optimal fit for their hand.

The methodology or logic behind the pattern sizing for the unisex glove is not evident to the user who only sees the size designated on the label of the glove. For example, for the person who tries on a glove size designated as “6”, they would likely assume that a size designated as “5” will have shorter fingers and a size designated as “7” will have longer fingers. However, as shown in the accompanying chart, the sizes of 4, 5, and 6 are indeed separated in finger length by approximately 6 millimeters per size. However, size 6, 7, and 8 are nearly the same in finger length and are mainly separated only by a difference in the hand circumference dimension.

It therefore becomes a trial and error process for the user to begin the selection process of choosing a proper fitting glove. And the process does not necessarily become easier or logical as they progress.

Other Military Glove Systems

In addition to the numerical sizing designators used in the military unisex dress glove, specification A-A-50386, another military glove style that utilizes a numerical size designator is the military Intermediate Cold Wet (ICW) glove referred to in specification MIL-G44419. In the ICW glove sizing system, an abstract sizing designation, and specifically, the numbers 1, 2, 3, 4, 5 and 6 are utilized for a standard glove size designation. These sizes represent gloves that fit an entire military population from the 5 percentile female to the 95 percentile male. Once again there is no direct quantified relationship between a users hand dimensions and the abstract descriptor. Also there is no size correlation between a Size 4 Unisex glove that typically would fit a Women's small hand and a Size 4 ICW glove that typically would fit a Men's large hand.

### SUMMARY

The present invention is an improved glove sizing system that incorporates two-dimensional dimensions of a human hand into a size designator of a standard glove size. The quantified size designator facilitates a quantified process for a user to select a proper glove size for best fit and tactility.

It is well known to a glove pattern designer that the two-dimensional measurements of a finger length and a palm width have a high correlation with all of the remaining measurements of the hand including a three-dimensional measurement such as a hand circumference. Therefore it is possible to create a set of glove patterns that are sized in relation to one or more of the two-dimensional hand measurements.

Thus, when the glove patterns are designed relative to a base dimension of a finger length and/or the palm width and when the sizes are designated with the same quantified dimension, the resultant gloves can be logically and quantifiably selected by the end user for proper size, fit and tactility.

Additionally, the glove size designations of the present invention refer to actual hand measurements that can be measured by a person with out using any specialized hand-measuring device.

Also, the size designations of the present invention are not abstract descriptors. Abstract descriptors are defined as words or alpha or numeric characters that describe a specific glove size, such that the abstract descriptors are either based on hand measurements made by a specialized hand-measuring device or the abstract descriptor has little correlation to a quantifiable base measurement and is generic. An example of a generic abstract descriptor would be the word “large”.

The glove sizing system of the present invention is a single sizing system that applies to both male and female hands.

Objects and Advantages

Accordingly the reader will see that the glove sizing system of the present invention addresses a need that users have for properly sized gloves, proper fitting gloves and tactile gloves. This is accomplished by introducing a quantified glove size designator that directly corresponds to a two-dimensional dimension of a human hand, such as an index finger length dimension. Additionally for greater sizing precision and selection precision, a second two-dimensional dimension of the human hand, such as an actual palm width dimension can be added to the glove size designation.

Alternatively, it may be desired to utilize a combination of a quantified size designation with an abstract size designation. An example would be the utilization of an index finger length dimension in the first part of the glove size designation and the utilization of an abstract palm width descriptor such as an alpha character of “N”, “R”, or “W” corresponding to “narrow”, “regular”, and “wide” respectively in the second part of the glove size designation.

An advantage of the present invention includes a size designation system based on measurements that can be performed by a person with out using any specialized hand-sizing tool or any specialized hand-measuring device. For example, a regular measuring scale or ruler can be used for the measurements.

Additionally, the glove sizing system of the present invention has an advantage of allowing a person selecting a proper glove size to logically chose, in a quantified manner, the glove size that corresponds to their measured hand dimension.

Additionally, the present invention allows a person selecting a glove to choose a best fitting glove by understanding the quantified relationship that exists between all of the standard glove sizes offered by the manufacturer. This quantified relationship amongst the various sizes is evident because the glove size designation corresponds directly to the dimensions of a human hand.

Additionally, the present invention allows a person selecting a glove to choose a glove that offers the best fit in terms of a specific attribute such as tactility This is possible when the glove size designation includes an index finger length dimension and the glove patterns are designed accordingly. In this case, the user selects a glove size designated with an index finger length dimension that corresponds quantifiably to the length dimension of his or her own index finger.

Additionally, the present invention has the potential to reduce the trial and error time required by a consumer when they are selecting a proper glove size. The reduced trial and error time is due to a quantified logic applied to the size designator. This logic facilitates a quantified process of selecting the proper glove size.

Additionally, the present invention is applicable to sales of gloves via e-commerce, catalogues and other similar mediums in which the consumer is not able to physically touch the glove product prior to purchasing. With the present invention, once the person knows one or two of their two-dimensional hand dimensions and that this dimension corresponds to the glove size, the choice of the proper size glove is more assured that when choosing a glove with an abstract descriptor such as “large”.

Additionally, the present invention provides a quantified and directly correlated process for a central procurement buyer to procure the proper quantity of each glove size once one or more easily measured hand dimensions of the users are known and kept in a database. The organizer of this data can select the proper glove size for each user by applying this data to a hand fit chart that accompanies the glove sizing system.

Additionally, the present invention becomes a self-policing policy for standardization amongst glove manufacturers because the size designator directly corresponds to the hand size of the glove it is intended to fit. This counters the common response by manufacturers that say their gloves run “large” or run “small” when a customer is dissatisfied with the fit of the gloves.

Additionally, the present invention aids the retailer by minimizing the times that customers need to take gloves out their packaging. This is because there is less need for the consumer to try on different sizes to select the best fitting glove.

It is, therefore, a purpose of the invention to provide a glove sizing system that facilitates a quantified process of selection for a user selecting a proper glove size for best fit and tactility.

### DRAWING FIGURES

FIG. 1 shows a table listing prior art glove sizing designations.

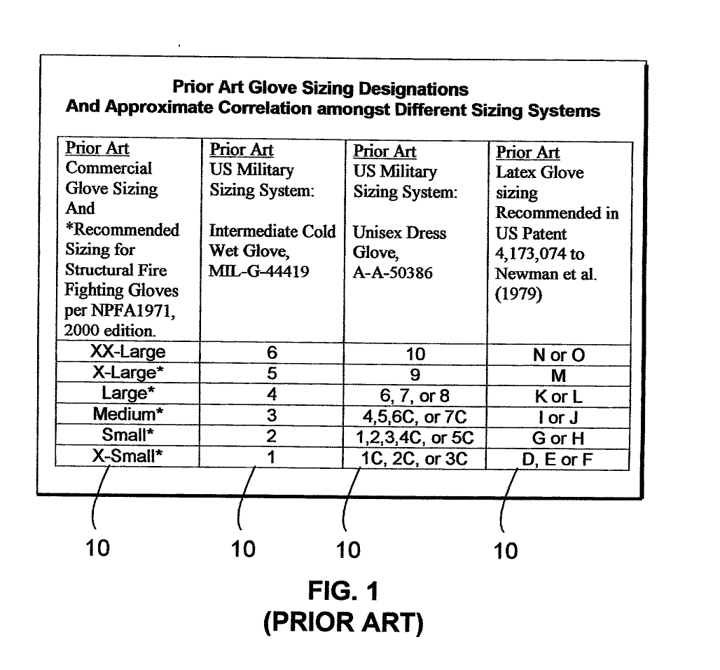

FIG. 2 is a chart showing the prior art US Military Unisex Dress Glove sizing designations vs. the relative pattern dimensions.

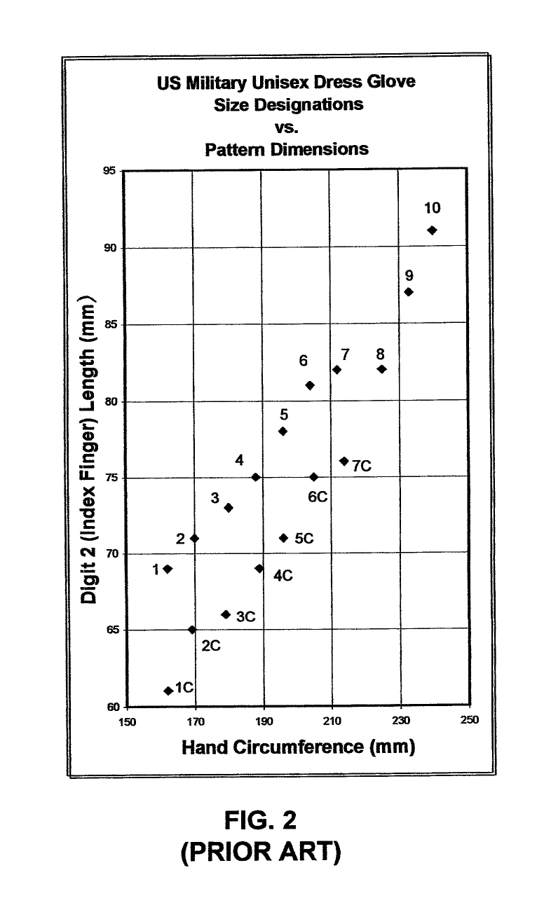

FIG. 3 is a printed Hand Sizing Tool illustrating one method for determining one's hand size and the respective glove size designation.

FIG. 4 is an illustration of a general method for measuring a pair of two-dimensional hand measurements.

FIG. 5 is a Hand Wear Fit Chart showing standard glove sizing designations and their correlated best-fit areas. The best-fit areas are defined by the dimension ranges for an index finger length and a palm width.

FIG. 6 shows a glove system containing three standard glove sizes labeled with their respective glove size designations.

### REFERENCE NUMERALS IN DRAWINGS

10 Abstract Sizing Designations

20 Hand Sizing Tool

22 Index Finger Guide

24 Index Finger Length Size

26 Palm Width Guide

28 Palm Width Size Designator

30 Pencil

32 Right Hand

34 Digit 1 Thumb

36 Digit 2 Index Finger

38 Digit 3 Middle Finger

40 Digit 4 Finger

42 Digit 5 Finger

44 Horizontal Line

46 Vertical Line

48 Index Finger Length Measurement

50 Index Finger Crease

52 Index Fingertip

54 Palm Width Measurement

56 Hand Wear Fit Chart Designator

58 Vertical Axis

60 Horizontal Axis

62 Best-Fit Area

64 Glove Size Designation

66 Glove System

68 Outer Glove Shell

70 Palm Portion

72 Back Portion

74 Fingerstall

76 Thumb stall

78 Opening

### DESCRIPTION OF INVENTION

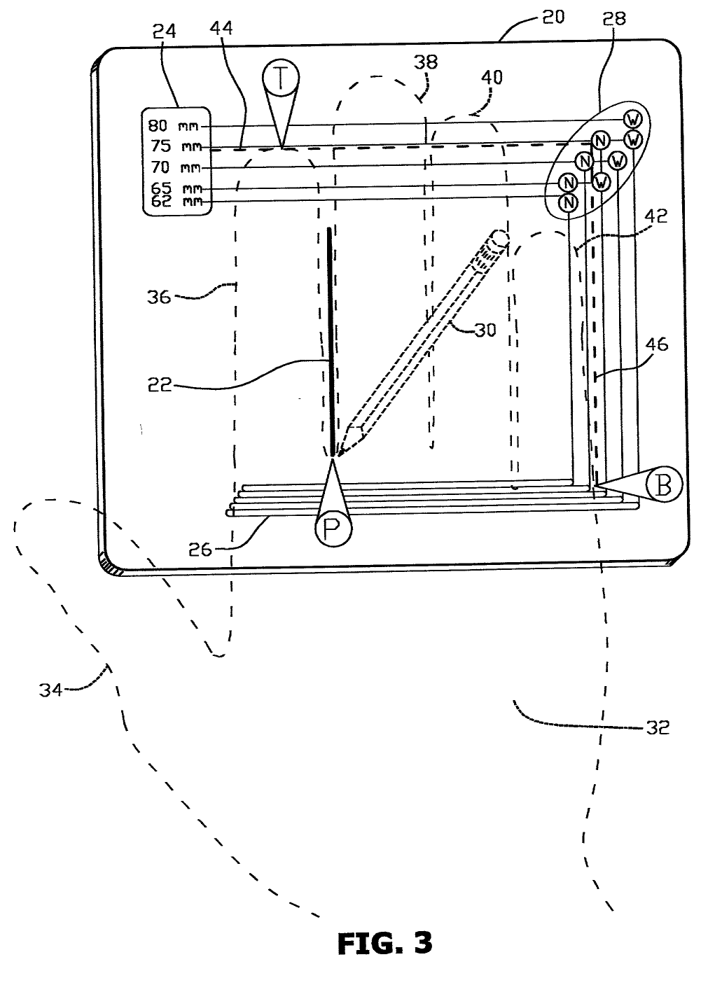

Description—FIG. 3—Preferred Embodiment

The preferred embodiment of measuring the hand dimensions and designating the sizes of the present invention is illustrated in FIG. 3. A hand-sizing tool 20 is printed on a rigid piece of cardboard. Although cardboard is used here as the medium for showing the hand sizing tool, other flat surfaces could be used for the sizing tool such as a sheet of paper or a computer monitor screen.

On the left side of the sizing tool 20 is an index finger guide 22. Located above and to the left of the guide 22 is an index finger length size designator 24. Located under the guide 22 is a palm width guide 26. The palm width guide 26 has several widths shown with a starting point increasingly to the left for larger palm widths. This is done to accommodate the wider index finger width associated with wider palms. Located to the right of the guide 22 is a palm width size designator 28.

The sizing tool shown in FIG. 3 is intended for use with a right hand. Although not shown, a mirror image of this chart can be produced for use with a left hand. It is recommended that the dominant hand of the user be measured.

The process for using the sizing tool begins by placing a pencil 30 or similar object centered at point P at the base of the index finger guide 22. The pencil functions as a stop. A right hand 32 is placed palm down with a digit #1 thumb 34 facing to the left. The web space between a digit #2 index finger 36 and a digit #3 middle finger 38 are butted against the stop at point P. Next, the index finger 36 and the middle finger 38 are positioned such that they align with the index finger guide 22. The remaining fingers including a digit #4 finger 40 and a digit #5 finger 42 are pressed together and a horizontal line 44 is traced from the tip T of the index finger to the index finger length size designator 24. Then a vertical line 46 is traced up from the base B of the digit #5 finger 42 to the palm width size designator 28.

A standard glove size is first determined by choosing a measurement from the index finger length size designator 24 that is closest to the horizontal line 44. If the horizontal line does not fall directly on a listed measurement, then the user will choose if they want the glove to be slightly shorter in length or slightly longer in length. If possible, it is recommended that a slightly shorter length be chosen for improved tactility, however this is a personal choice.

Secondly, the user will determine the glove size for the palm width by choosing a measurement from the palm width size designator 28 that is closest to the intersection of the vertical line 46 and the printed horizontal line extending from the size chosen for the index finger length. Again, similarly to choosing the preferred finger length size, if the vertical line does not fall directly on the listed palm width measurement, then the user will choose if they want the glove to fit slightly snugger or slightly looser. Because the fit in the palm width does not effect tactility as greatly as the fit in the index finger length, it is recommended to choose a palm width fit that will allow for easy donning and doffing of the glove. Therefore it is recommended that the person choose a palm width size designation that is directly on the vertical line or the next size larger located to the right of the vertical line.

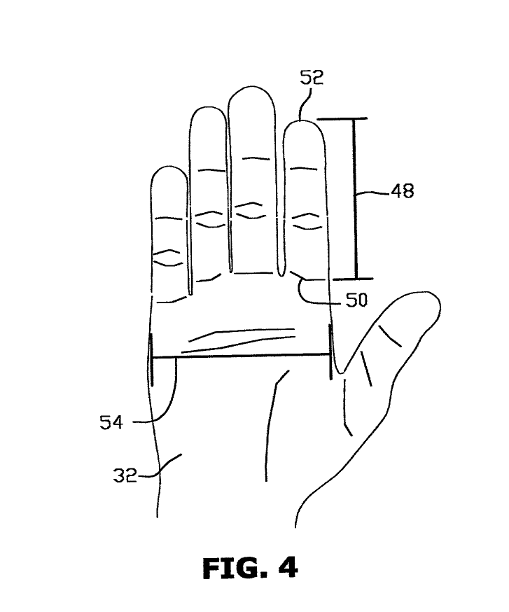

Description—FIG. 4—Alternative Method for Measuring the Hand

FIG. 4 illustrates an alternative method for measuring the hand and applying hand dimensions directly to the glove sizing system of the present invention. No special hand-measuring devices or special hand sizing tools are required.

Shown in FIG. 4 is a right hand 32 with the palm side facing towards the viewer. An index finger length measurement 48 is shown as measured from an index finger crease 50 to an index fingertip 52. A palm width measurement 54 is shown as measured across the palm of the hand at the base of the fingers.

The index finger length measurement 48 is made by positioning a ruler, scale or calipers at the index finger crease 50 and measuring to the index fingertip 52. The preferred scale reading is in metric units. Secondly the palm width measurement 54 is made in a similar manner by measuring the palm width at the base of the fingers.

A user then matches these dimensions as close as possible to a standard glove size labeled according to the present invention with an index finger length dimension and/or a palm width dimension.

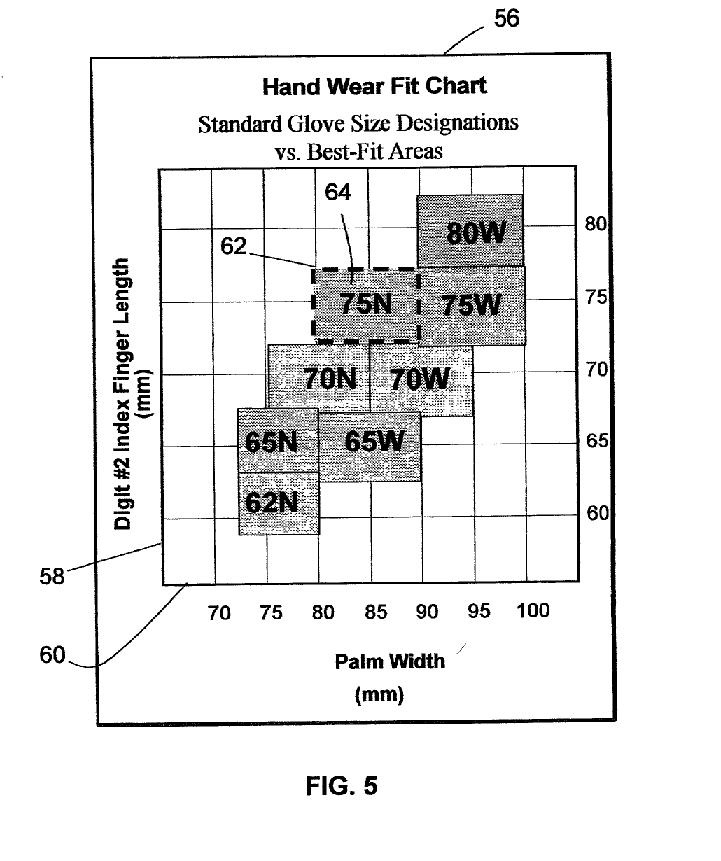

Description—FIG. 5—Hand Wear Fit Chart of Preferred Embodiment Showing Standard Glove Sizes

FIG. 5 shows a hand wear fit chart 56 containing a vertical axis 58 for a Digit #2 Index Finger Length and a horizontal axis 60 for a Palm Width. A best-fit area 62 is shown as a dashed heavy line boxed area. A glove size designation 64 is depicted as “75N”.

The best-fit area 62 is a graphical representation of an area defining particular sets of hand dimensions. The glove size designation 64 is intended to fit any set of hand dimensions that can be graphed within the best-fit area. As can be seen in the chart, the glove size designation 64 corresponds quantitatively to the index finger length dimension, and qualitatively to the palm width dimension. The “75” refers to an index finger length dimension of “75 millimeters”. The qualitative designation of “N” for the palm width measurement was obtained from the hand-sizing tool 20, shown in FIG. 3, and the corresponding palm width size designator 28. The “N” refers to “Narrow”. Alternatively, if greater precision is desired in regards to the palm width, the palm width size designator can be quantitatively designated as the actual hand measurement dimension.

Although boxed shaped areas are utilized for the best fit area in the handwear fit chart, one could also utilize overlapping ellipses or circles to indicate the best-fit size area.

The best fit areas and the accompanying glove size designations depicted in the hand wear fit chart can be designed to fit a majority of the male and female population. Typically this is referred to as fitting the 5 percentile female to the 95 percentile male. Accordingly, the sizing system of the present invention is applicable to both the female and male population.

The hand wear fit chart of the preferred embodiment was designed with advance knowledge of many factors including the following list.

1. The two-dimensional hand dimensions of the population to be fitted, specifically the index finger length and the palm width.

2. The types of materials to be used in the construction of the glove and specifically the amount of stretch resulting in the final glove composite.

3. The tasks to be performed by the user and the desired fit to be achieved specifically regarding tactility.

4. The economics controlling the number of standard glove sizes that are practical.

These factors were considered when designing the best-fit areas shown in the hand wear fit chart 56. It can be observed that amongst the standard glove sizes listed and the respective best-fit areas, the dimension range for the index finger length is less than the dimension range for the palm width. These ranges were designed intentionally. The index finger length range was designed to provide a relatively close fitting glove at the fingertips for the purpose of minimizing any loss of tactility due to fit. The palm width range was designed with a larger tolerance because the same level of precision was not required to provide a satisfactory fit to the user.

In the hand wear fit chart 56, the best-fit areas are rectangular shapes defining the dimension ranges for the hand measurements.

Once the dimension range for a best-fit area was defined, it was then necessary to distribute a multiple of best-fit areas over the range of hand dimensions comprising the target end user group. These areas were distributed such that the standard glove sizes would provide a satisfactory fit to a majority of the user population including the 5 percentile female to the 95 percentile male.

Ideally the number of best-fit areas required to cover the target end user group coincide with the economics controlling the number of standard sizes that is practical. If this is not the case then compromises must be made in defining the dimension range for an appropriate fit such that the best-fit areas are made slightly larger to minimize the number of standard sizes.

Based on the designed best-fit areas, the glove manufacturer then produces standard glove sizes with pattern dimensions correlated to the two-dimensional hand dimension noted in the glove size designation.

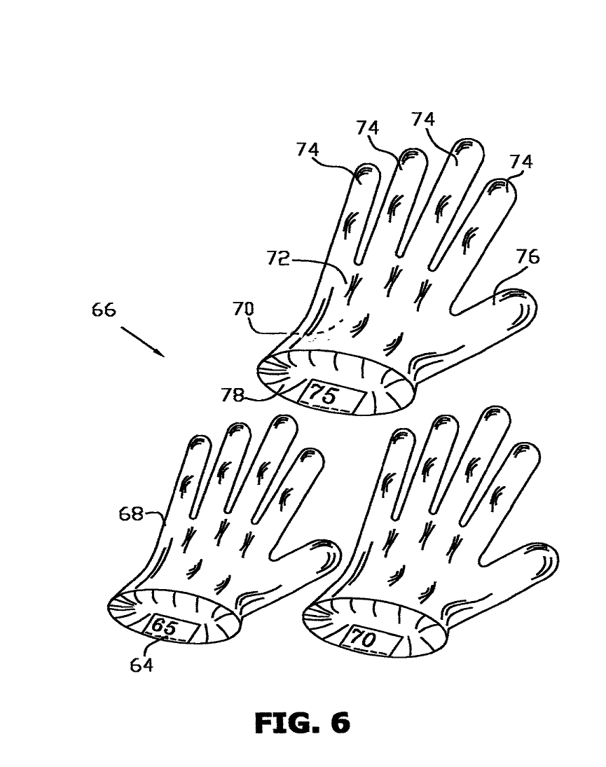

Description—FIG. 6—Glove System

FIG. 6 illustrates generally at 66 a glove system. As used herein, a “glove system” means a plurality of gloves made in a multiple of standard glove sizes. The gloves of the shown glove system may perform the function of a stand-alone glove or they may be just one component of a multiple component glove. The components of the glove may include any of the following: an outer glove shell 68, a barrier insert for waterproofing, (not shown), and an insulation and/or an inner lining, (not shown).

The gloves illustrated in the glove system 66 may be produced from any suitable material, such as but not limited to knit, woven or nonwoven materials, leather, composite fabrics, rubber or any other suitable material. The gloves may be patterned in accordance with any suitable pattern, such as but not limited to, a flat two-dimensional pattern, the Clute Cut Pattern, Gunn Cut Pattern, Forchette Pattern, or the Star pattern for example. Additionally the gloves may be made from a molded pattern such as utilized in making rubber gloves.

As best seen by reference to FIG. 6, the outer shell glove 68 may include any of the following: a palm portion 70, a back portion 72, finger stalls or passageways 74, a thumb stall or passageway 76, and an opening 78 facing inwardly of the wearer. Although the outer glove shell 68 is illustrated as a conventional glove, in the sense that it includes an individual finger stall for each finger of a human hand and a thumb stall, the teachings of the present invention may be applied to other gloves having less than four finger stalls but at least one.

Each of the gloves shown in the glove system 66 is labeled with a glove size designation 64.

When the user is presented with making a selection of the proper glove size from the given glove system, they can use a quantitative and logical selection process. With prior knowledge of their own two-dimensional hand dimensions, they will choose a glove labeled with a glove size designation the most closely matches their own hand dimension. This selection process can be performed without the physical glove sample present. For example this quantified selection process can occur when the glove is selected from a catalogue or web site.

Referring to FIG. 5, additionally the user can utilize the hand wear fit chart 56, to observe how close their hand dimensions are to the boundary area defined by the best-fit area 62. If by chance, their hand dimensions fall directly on the boundary line for the index finger length, then they can logically decide to choose a standard glove size that is slightly longer and thus looser at the fingertip or they can choose a standard glove size that is slightly shorter at the fingertip for minimizing any loss of tactility. A similar logic can be applied if their hand dimensions fall directly on a boundary line for the palm width. They can logically decide if they desire a slightly tighter fit or a slightly looser fit and thus choose the appropriate standard glove size.

Ramifications & Scope

Although the description above contains much specificity, these should not be construed as limiting the scope of the invention but as merely providing illustrations of some of the presently preferred embodiments of this invention.

For example, while the metric scale and specifically millimeters is the preferred unit of measure to incorporate into the size designation of the index finger length, other units of measure such as the English units of inches could be used.

Although the specific glove sizing system of this invention is specifically focused on tactility and consequently the size designation directly relates to the index finger length dimension, the same logic could be applied to other dimensions of the hand if a specific advantage can be gained by specifying the dimensional property of the specific hand dimension as the size designator.

The glove sizing system of the present invention can be utilized on any coverings for the hands such as gloves or mittens. The term “glove” is intended to refer to any hand covering having at least one fingerstall and a thumb stall.

The glove sizing system of the present invention is applicable to other types of gloves in addition to the military and fire gloves mentioned, such as snow sport gloves, healthcare gloves, law enforcement gloves, industrial protective gloves etc.

Thus the scope of the invention should be determined by the appended claims and their legal equivalents, rather that by the examples given.

Having described the invention, what is claimed is:

1. A glove sizing system to aid one in the tasks of designing, manufacturing, labeling, and selecting comprising:
a. a glove size designation,
b. a plurality of standard glove sizes,
c. each of said standard glove sizes pertaining to a glove having at least one fingerstall, a thumb stall, a palm portion, and a back portion,
d. said glove intended to fit a human hand,
e. a means for measuring a plurality of two-dimensional dimensions of said human hand,
f. each of said standard glove sizes having a said glove size designation comprising at least one of said two-dimensional dimensions,
whereby said glove size designation facilitates a quantified and logical process for a user to select a proper glove size for best-fit and tactility without the necessity of using a special hand-measuring device.

2. The glove sizing system of claim 1 wherein a unit of measure for said two-dimensional dimensions is millimeters.

3. The glove sizing system of claim 1 wherein a unit of measure for said two-dimensional dimensions is inches.

4. The glove sizing system of claim 1 wherein said means for measuring said two-dimensional dimensions of said human hand include an index finger length dimension and said standard glove sizes comprise said glove size designations comprising said index finger length dimension.

5. The glove sizing system of claim 1 wherein said means for measuring two-dimensional dimensions of said human hand include a palm width measurement and said standard glove sizes comprise said glove size designations comprising said palm width measurement.

6. A glove system comprising:
a. a plurality of gloves comprising,
b. at least one fingerstall, a thumb stall, a palm portion, and a back portion and,
c. a glove size designation and,
d. a plurality of standard glove sizes,
e. said gloves intended to fit a human hand,
f. a means for measuring a plurality of two-dimensional dimensions of said human hand,
g. each of said standard glove sizes having a said glove size designation comprising at least one of said two-dimensional dimensions,
whereby said glove size designation facilitates a quantified and logical process for a user to select a proper glove size for best-fit and tactility without the necessity of using a special hand-measuring device.

7. The glove system of claim 6 wherein a unit of measure for said two-dimensional dimensions is millimeters.

8. The glove system of claim 6 wherein a unit of measure for said two-dimensional dimensions is inches.

9. The glove system of claim 6 wherein said means for measuring said two-dimensional dimensions of said human hand includes an index finger length dimension and said glove size designation comprises said index finger length dimension.

10. The glove system of claim 6 wherein said means for measuring two-dimensional dimensions of said human hand include a palm width measurement and said glove size designation comprises said palm width measurement.

11. A method for designating a glove sizing system comprising a plurality of gloves, each of said gloves comprising at least one finger stall, a thumb stall, a palm portion, and a back portion, comprising the steps of:
a. providing a measurement means which a human can use to measure a plurality of two-dimensional dimensions of a human hand, and
b. designating said standard glove sizes with at least one of said two-dimensional dimensions of said human hand,
whereby said designation facilitates a quantified and logical process for a user to select a proper glove size for best-fit and tactility without the necessity of using a special hand-measuring device.

12. The method of claim 11 wherein a unit of measure for said two-dimensional dimensions is millimeters.

13. The method of claim 11 wherein a unit of measure for said two-dimensional dimensions is inches.

14. The method of claim 11 wherein said two-dimensional dimensions include an index finger length dimension and said standard glove sizes comprise said index finger length dimension.

15. The method of claim 11 wherein said two-dimensional dimensions include a palm width dimension and said standard glove sizes comprise said palm width dimension.
